(19) United States
(12) Patent Application Publication
(10) Pub. No.: US 2020/0060582 A1
(43) Pub. Date: Feb. 27, 2020

(54) SYSTEM FOR DETECTING DISTORTION OF BODY

(71) Applicant: Shoichi NAKAMURA, Nagano-ken (JP)

(72) Inventor: Shoichi NAKAMURA, Nagano-ken (JP)

(73) Assignees: Shoichi NAKAMURA, Nagano-ken (JP); ACP JAPAN CO., LTD., Tokyo (JP)

(21) Appl. No.: 16/110,510

(22) Filed: Aug. 23, 2018

(51) Int. Cl. A61B 5/11; A61B 5/107; A61B 5/00; A61B 5/0205

(52) U.S. Cl. CPC A61B 5/1116; A61B 5/1077; A61B 5/6803; A61B 5/1121; A61B 5/02055; A61B 5/02438; A61B 5/7278; A61B 2562/0219; A61B 2560/0475; A61B 2562/227; A61B 5/1114

(57) ABSTRACT

It is an object to provide a body distortion detection system for detecting distortion of the body from postures of natural manners in daily action of an examinee. A body distortion detection system includes a posture detection apparatus mounted on the body of an examinee, and a distortion determination apparatus. Upon acquiring acceleration information periodically from an acceleration sensor mounted on a part for detecting a movement of the body of the examinee, the posture detection apparatus calculates a moving distance of the part from the acquired acceleration information to obtain coordinates of the part in a computation processing section. Then, the apparatus stores coordinates obtained by the computation processing section point by point in memory. The distortion determination apparatus determines distortion of the body, based on a tilt angle of the part calculated from a series of coordinates read by connection to the memory.

### TECHNICAL FIELD

The present invention relates to a body distortion detection system for detecting distortion of a posture by acquiring data of the posture of a user over a long time.

### BACKGROUND ART

Distortion of the body occurring from a lifestyle habit and a habit of personal manner becomes causes of various bad conditions such as shoulder discomfort, low back pain, swelling and headache. Accordingly, by detecting a part and cause of distortion of the body, and correcting to a proper posture to improve the distortion, it is possible to keep the body in excellent condition.

In Japanese Patent Application Publication No. 2009-219622 (Patent Document 1) is disclosed a posture evaluation apparatus for evaluating a posture of a user to output an evaluation result, based on a tilt of a hold portion held with both hands by a user, and a position of the center of gravity of a load acting on a footstool which the user gets on with both legs, in order to detect distortion of the body.

Further, in Japanese Patent Application Publication No. 2010-207399 (Patent Document 2) is disclosed a detection system for determining postures of right and left arms from data obtained by measuring the three-dimensional posture with first sensor and second sensor attached to right and left upper arms of the body, and determining a strong part of a muscle of the upper body corresponding to a difference in posture between the right and left arms to detect distortion.

### DISCLOSURE OF INVENTION

### Problems to be Solved by the Invention

However, to detect distortion of the body, the apparatus disclosed in Patent Document 1 uses measurement results obtained in performing predetermined action using a particular measurement instrument provided with the hold portion and footstool, and is not to measure distortion of the body from daily action of an examinee.

Further, the apparatus disclosed in Patent Document 2 is to detect distortion by detecting the strong part of the muscle corresponding to a difference in posture between the right and left arms, there is a difference between muscle strengths of the right and left arms, and it is general that the dominant arm is stronger. Accordingly, it is not possible to always correctly detect distortion from the difference between right and left muscle strengths.

In order to solve the above-mentioned problems, it is an object of the present invention to provide a body distortion detection system for detecting distortion of the body from postures of natural manners in daily action including work postures in a job of an examinee, without using any particular measurement instrument.

### Means for Solving the Problem

In order to attain the above-mentioned object, the body distortion detection system according to the present invention is a body distortion detection system including a posture detection apparatus mounted on the body of an examinee, and a distortion determination apparatus, and is characterized in that the posture detection apparatus is provided with an acceleration sensor mounted on a part for detecting a movement of the body of the examinee, a computation processing section for obtaining coordinates of the part, by calculating a moving distance of the part from acceleration information acquired periodically from the acceleration sensor, and a memory for storing the coordinates obtained by the computation processing section point by point, and that the distortion determination apparatus determines distortion of the body, based on a tilt angle of the part calculated from a series of the coordinates read from the memory.

Then, the distortion determination apparatus is characterized by reading the coordinates from the memory by USB connection with the posture detection apparatus. Accordingly, when the posture detection apparatus is connected to the distortion determination apparatus by USB, the computation processing section receives power supply from the distortion determination apparatus by a bus power function, while receiving references to data stored in the memory from the distortion determination apparatus by a mass storage function.

The memory may be comprised of a card type of flash memory capable of being removed from the posture detection apparatus to connect to the distortion determination apparatus.

Then, the computation processing section and the memory may be packaged with the acceleration sensor to be mounted on the part.

The acceleration sensor is worn by mounting on the head of the examinee or binocular loupes worn by the examinee.

### Advantageous Effect of the Invention

According to the present invention, the examinee wears the acceleration sensor to detect the movement of the body, the daily posture is thereby measured without the examinee taking a particular posture or action, and therefore, it is possible to correctly determine the distortion.

### BRIEF DESCRIPTION OF DRAWINGS

FIG. 1 illustrates a schematic configuration of a body distortion detection system according to this Embodiment of the present invention, in a block diagram;

FIG. 2 illustrates a view to explain an ideal posture that does not cause distortion of the body when a dentist provides treatment to a patient;

FIG. 3 shows a flowchart to explain a processing procedure of a posture detection apparatus of the body distortion detection system according to this Embodiment of the invention;

FIG. 4 illustrates an explanatory diagram illustrating three-dimensional coordinates of a measurement part associated with a movement of an examinee;

FIG. 5 is an explanatory diagram illustrating coordinate positions of each point of FIG. 3 in XY two-dimensional coordinate system;

FIG. 6 is an explanatory diagram illustrating coordinate positions of each point of FIG. 3 in ZY two-dimensional coordinate system;

FIG. 7 is an explanatory diagram illustrating coordinate positions of each point of FIG. 3 in XYZ three-dimensional coordinate system;

FIGS. 8A and 8B are explanatory diagrams respectively illustrating the three-dimensional coordinates of FIG. 7 in the XY two-dimensional coordinate system and ZY two-dimensional coordinate system;

FIG. 9 is an explanatory diagram illustrating coordinate positions of each point in the XYZ three-dimensional coordinate system with a reference position of the measurement part as the origin point;

FIGS. 10A and 10B are explanatory diagrams respectively illustrating the three-dimensional coordinates of FIG. 9 in X′Y two-dimensional coordinate system and X″Y two-dimensional coordinate system;

FIG. 11 illustrates a conceptual explanatory diagram of a memory format of memory;

FIG. 12 illustrates a schematic explanatory diagram of a screen for displaying distortion determination results by a distortion determination apparatus of the body distortion detection system according to this Embodiment of the invention;

FIG. 13 illustrates a schematic explanatory diagram of a screen for displaying, in time series, distortion determination results by the distortion determination apparatus of the body distortion detection system according to this Embodiment of the invention;

FIG. 14 illustrates an explanatory diagram of a configuration with an accelerator sensor mounted on binocular loupes; and

FIG. 15 is an explanatory diagram illustrating three-dimensional coordinate positions of a measurement part in a posture detection apparatus provided with two acceleration sensors.

### MODE FOR CARRYING OUT THE INVENTION

Embodiments of the present invention will be described below with reference to drawings.

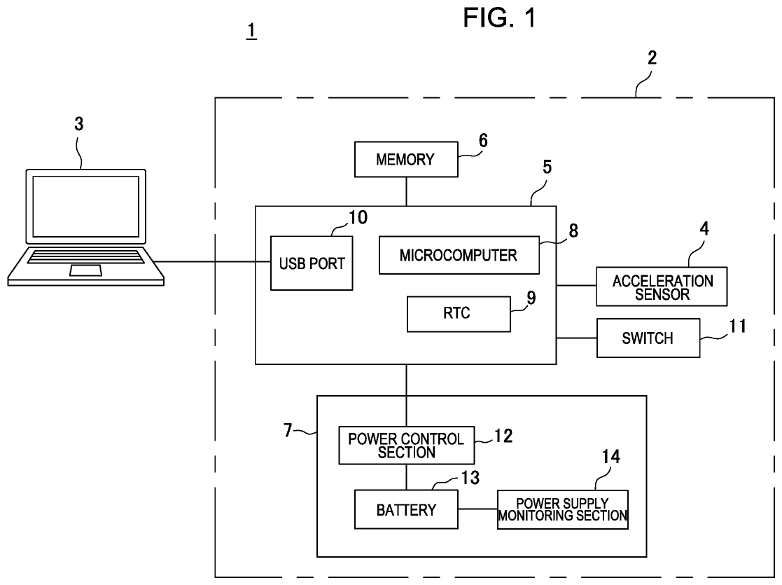

FIG. 1 illustrates a schematic configuration of a body distortion detection system 1 according to this Embodiment, in a block diagram. As shown in FIG. 1, the body distortion detection system 1 is comprised of a posture detection apparatus 2 mounted on the body of a user to measure changes in posture of the user over a long time, and an information processing apparatus 3. The information processing apparatus 3 executes a program of distortion determination, thereby functions as a distortion determination apparatus, and determines distortion of the body of the user, by performing computation processing of measured data from the posture detection apparatus 2.

The posture detection apparatus 2 is provided with a triaxial acceleration sensor 4, computation processing section 5, memory 6, power supply section 7 and switch 11.

When the posture detection apparatus 2 is mounted on a part for detecting a movement of the body of an examinee, the triaxial acceleration sensor 4 is a position detection sensor for detecting position information of the part respectively with accelerations in mutually orthogonal three-axis (X-axis, Y-axis, Z-axis) directions to output. Thus, in this example, the triaxial acceleration sensor 4 is used as the position detection sensor, and an angular velocity sensor may be used.

The computation processing section 5 is comprised of a control board provided with a microcomputer 8, real-time clock circuit (RTC) 9, and USB (Universal Serial Bus) port 10. Then, when the microcomputer 8 acquires an acceleration of each of the X axis, Y axis and Z axis from the acceleration sensor 4, the microcomputer 8 performs time integration on the accelerations, thereby calculates respectively moving distances in three axes, and identifies a spatial position at this point.

With an initial position in the X axis, Y axis and Z axis of the acceleration sensor 4 as the origin point, the microcomputer 8 calculates moving distances x, y, and z in three axes from the origin point, and thereby obtains coordinates in three-dimensional space at this point. Then, the microcomputer 8 associates the obtained spatial coordinates with time information output from the RTC 9 at this point to store in the memory 6. In this case, for example, the computation processing section 5 outputs the coordinates in space and time information to the memory 6 every second.

The memory 6 is a type of memory capable of performing erasing and writing of data freely where the contents are not lost when power supply is disconnected, and flash memory is suitable. The posture detection apparatus 2 is mounted on a user for 24 hours at the maximum, and in the case of configuring to detect changes in posture during the period, the memory 6 is provided with storage capacity enough to store 86,400 (seconds) coordinates and time information sent from the computation processing section 5

The USB port 10 connects the posture detection system 1 to the information processing apparatus 3 by USB. When the posture detection apparatus 2 is connected to the information processing apparatus 3 by USB, the computation processing section 5 receives power supply from the information processing apparatus 3 by the bus power function, while receiving references to data stored in the memory 6 from the information processing apparatus 3 by the mass storage function. In addition, as the memory 6, a card type of flash memory may be used which is capable of being removed from the posture detection apparatus 2 to connect to the information processing apparatus 3.

The power supply section 7 is provided with a power supply control section 12, battery 13, and voltage monitoring section 14. The power supply control section 12 supplies power of the battery 13 to the computation processing section 5, while controlling to charge the battery 13 by the bus power, when the body distortion detection system 1 is connected to the information processing apparatus 3 by USB. The voltage monitoring section 14 monitors the voltage of the battery 13, and when the voltage is decreased to a predetermined level, lights an indicator to display a warning.

As clarified later, the switch 11 is operated in making setting of a reference position.

A correct posture is kept by muscle strength of the body, and during daily action, particularly, when a person performs work, the person tends to take an easy posture. However, the easy posture is a posture such that the muscle strength is defeated by gravity, and when a state in which the correct posture is not kept continues for many hours, such a state is a cause of generating distortion of the body.

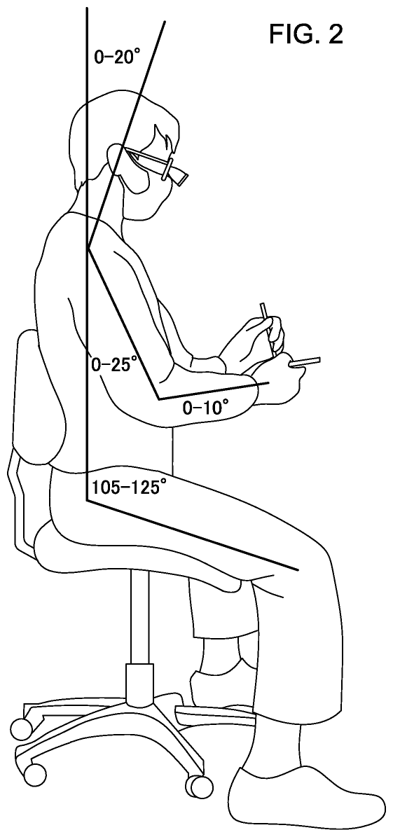

As an example of the correct posture at the time of work, described is a posture when a dentist provides treatment to a client. As shown in FIG. 2, it is proper that a tilt of the head with the neck as a supporting point with respect to the center axis of the body kept vertical is in a range from 0 degree to 20 degrees forward, and that a moving angle of each of elbows of both arms with the shoulder as a supporting point is in a range from 0 degree to 25 degrees forward. In this case, when the head is at an angle of 25 degrees or more at the maximum, the center axis of the body is curved to be round shoulders, and such an angle is a cause of distortion of the body.

Further, it is considered at this point that a rising angle of the forearm from the horizontal direction is suitably in a range from 0 degree to 10 degrees, and that an angle of the axis line of the upper thigh part with respect to the center axis of the body is suitably in a range from 105 degrees to 125 degrees. Then, as a result of concentration on the treatment, when a posture that a balance of right and left of the body is lost e.g., a state that the neck is inclined to one of the right and left is continued for many hours, such a state is a cause of putting a load on the cervical spine.

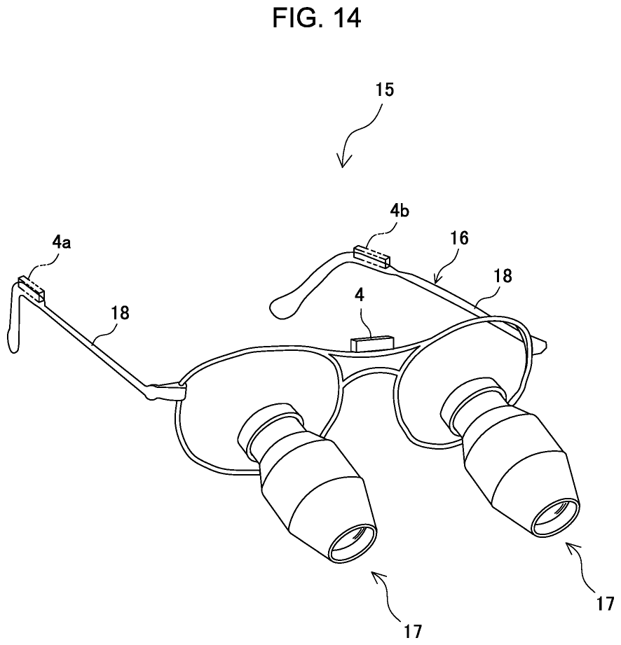

In such treatment work by the doctor, it is possible to use the distortion detection system 1 according to the present invention in measuring a forward tilting angle and tilt to the right or left of the head of the doctor during the treatment. In this example, as shown in FIG. 14, the acceleration sensor 4 is attached to a frame 16 of the binocular loupes 15 worn by the doctor to use. In the binocular loupes 15 shown in the figure, the acceleration sensor 4 is attached to an upper portion of the bridge at the center of the frame 16. In addition, the binocular loupes 15 are widely used, as a means for enlarging a local visual object at hand (procedure portion) with binocular loupes bodies 17 to visually identify.

Then, the computation processing section 5, memory 6, power supply section 7 and switch 11 are stored in a case as a control unit, and are mounted on the body of an operator to be held. An operation section of the switch 11 is provided on the frontside of the case. Thus, in this example, the acceleration sensor 4 is separated from the posture detection apparatus 2, and is mounted on the binocular loupes 15, and without separating, it is possible to actualize the sufficiently miniaturized posture detection apparatus 2. In such a posture detection apparatus 2, the apparatus may be mounted on a headband or medical cap to be worn by the doctor. Further, also in this case, the posture detection apparatus 2 may be configured so that only the acceleration sensor 4 is mounted on a headband or medical cap.

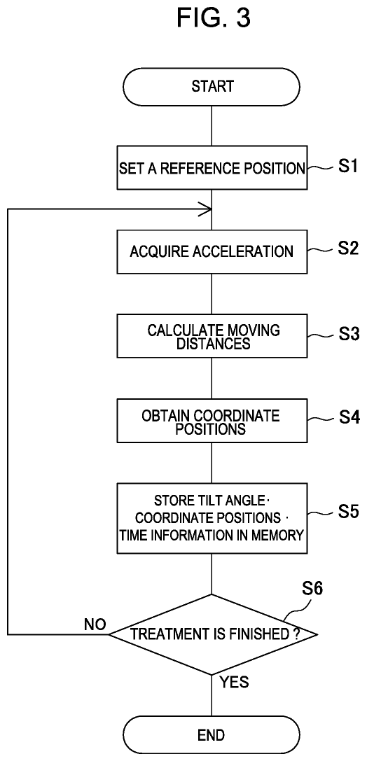

Described is a method of measuring a tilt of the head when the doctor provides treatment. FIG. 3 shows a flow of measuring a tilt of the head.

First, an examinee (doctor) straightens his/her posture so that center lines of the head and back are in the same vertical line in a state in which the binocular loupes 15 are worn on the face, and takes a posture to make visual observation in the horizontal direction. When the switch 11 is operated in this state, the microcomputer 8 makes setting of a reference position (step S1).

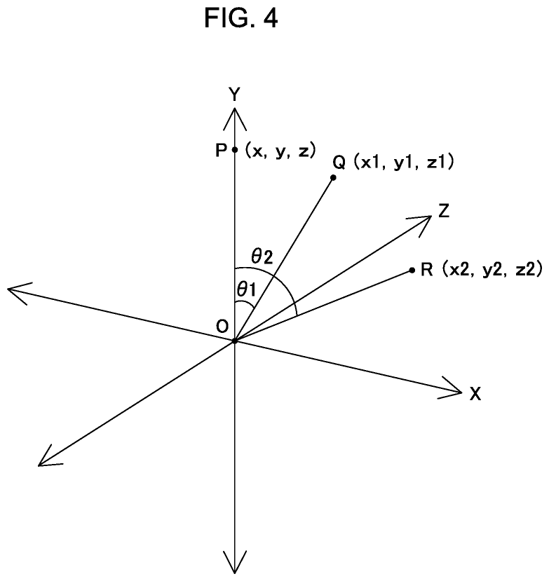

FIG. 4 illustrates a position of the head in three-dimensional coordinates, the origin point O in space is a position of the neck as a supporting point in tilting the head forward, backward, leftward or rightward, and a point P is a center position of the head of the examinee when the posture is straightened in the perpendicular direction that is the Y-axis direction and the line of sight is directed in the horizontal direction that is the X-axis direction. The coordinates (x, y, z) of the point P are the reference position in subsequently detecting a displacement when the examinee moves the head with the neck as a supporting point.

After setting the reference position, the microcomputer 8 acquires respective accelerations in three axes from the acceleration sensor 4 (step S2), integrates the acquired accelerations with respect to time, and calculates moving distances of the acceleration sensor 4 in the three-axis directions (step S3).

Next, the microcomputer 8 obtains the coordinates based on the calculated moving distance (step S4). Then, when the coordinates are obtained, the microcomputer 8 calculates a forward tilt angle of the acceleration sensor 4, and stores the tilt angle, the coordinates and time information output from the RTC 9 at this point in the memory 6 (step S5).

Then, the microcomputer 8 determines whether or not the treatment by the examinee is finished (step S6). When the treatment by the examinee is finished, the switch 11 is operated again. Accordingly, for a period during which the switch 11 is not operated again (“NO” in step S6), the microcomputer 8 performs processing of from step S2 to step S5. At this point, the microcomputer 8 acquires the acceleration from the acceleration sensor 4 for each second in step S2, and repeats the processing up to step S5.

Accordingly, after setting the reference position, when the line-of-sight direction is shifted to a patient positioned below from the horizontal direction in order for the examinee to start treatment, the microcomputer 8 integrates the acceleration acquired from the acceleration sensor 4 with respect to time at this point, calculates respective moving distances in the three-axis directions from the point P, and thereby computes coordinates (x1, y1, z1) of a point Q in which the head is positioned. Accordingly, a tilt angle θ1 of the head is calculated from numeric values of x1 and y1.

Further, when the examinee tilts the head from the position of the point Q to a position of a point R, at this point, since moving distances in three axes calculated by integrating the acceleration acquired from the acceleration sensor 4 with respect to time are displacement amounts from the point Q, the microcomputer 8 coverts to moving distances from the reference position P, and determines coordinates (x2, y2, z2) of the point R.

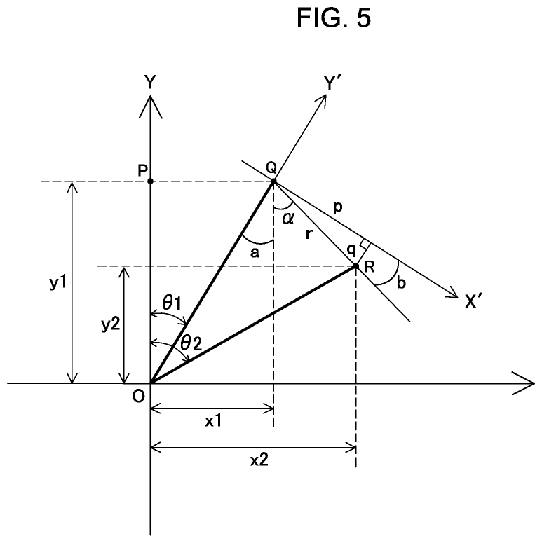

Described is a method of converting coordinates of the point (the next point) subsequent to moving as a point that is directly moved from the reference position P when the measurement part (head) thus moves from some point to the next point. Herein, on the assumption that the movement of the head of the examinee from the point P to the point Q and point R is only in the back-and-forth direction (X-axis direction), and that the movement in the right-and-left direction (Z-axis direction) does not exist, the method will be described in the two-dimensional coordinate system in FIG. 5. In FIG. 5, the straight line which extends from the origin point O and passes through the point Q is assumed to be a Y′ axis, and the straight line which passes through the point Q and is orthogonal to the Y′ axis is assumed to be an X′ axis. Herein, it is assumed that the tilt movement from the point Q to the point R includes a moving distance p in the X′-axis direction, and a moving distance q in the Y′-axis direction.

As shown in FIG. 5, the angle θ1 which the Y axis forms with the Y′ axis i.e. an angle a is arc tan (x1/y1). An angle b which the straight line joining the point Q and the point R forms with the X′ axis is arc tan (q/p). When an angle a which the straight line joining the point Q and the point R forms with the Y axis is added to the angle a and the angle b, the resultant is a right angle, and therefore, the angle α is obtained from the equation α=90°-a-b. Then, from the angle α and a distance r (=(p²+q²)^{1/2}) between the point p and the point R obtained from numeric values of p and q, coordinates (x1+r×sin α, y1−r×cos α) of the point R are calculated. Accordingly, from these calculated values, detected is a forward tilt angle θ2 at the time the head is positioned in the point R.

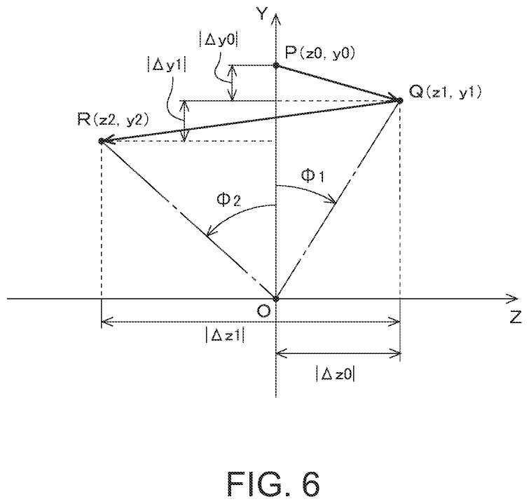

Also when the head is tilted only in the right-and-left direction, as in the explanation described above in association with FIG. 5, ZY coordinates of the head subsequent to moving are calculated from moving distances in the Z-axis direction and Y-axis direction of the acceleration sensor 4, and it is possible to obtain a tilt angle in the right-and-left direction with reference to the reference position P. Further, according to this Embodiment, the tilt angle of the head in the right-and-left direction is obtained as shown next, and will be described below, using the ZY two-dimensional coordinate system shown in FIG. 6.

In FIG. 6, the point Q is a position of the head tilted in the right-and-left direction from the reference position P, and the point R is a position of the head tilted in the right-and-left direction from the point Q. The tilt angle will be expressed, assuming that a tilt (clockwise) in the Z axis+direction from the Y axis with the origin point O as the center is the positive (+) direction, and that another tilt (counterclockwise) in the Z axis−direction is the negative (−) direction. In FIG. 6, after tilting the head from the reference position P to the point Q in the positive direction, the head is tilted from the point Q to the point R in the negative direction.

When it is assumed that coordinates of the point Q are (z1, y1), it is possible to obtain values of z1 and y1, by integrating accelerations in the Z-axis and Y-axis directions respectively acquired from the acceleration sensor 4 with respect to time, and thereby calculating moving distances Δz0 and Δy0 from the reference position P to the point Q in the Z-axis and Y-axis directions to add to the coordinates (z0, y0) of the reference position P. Herein, for the moving distance of the head, each of the Z-axis+direction and Y-axis+direction is assumed to be the positive direction. The tilt angle of the head in the point Q is assumed to be ϕ1 (positive in this Embodiment). The tilt angle ϕ1 is obtained by arc tan (z1/y1)=arc tan {Δz0/(y0+Δy0)}.

Next, when it is assumed that coordinates of the point R are (z2, y2), it is possible to obtain values of z2 and y2, by integrating accelerations in the Z-axis and Y-axis directions respectively acquired from the acceleration sensor 4 with respect to time, and thereby calculating moving distances Δz1 and Δy1 from the point Q to the point R in the Z-axis and Y-axis directions to add to the coordinates (z1, y1) of the point Q. Accordingly, it is obtained that z2=z1+Δz1, and that y2=y1+Δy1. The tilt angle of the head in the point R is assumed to be ϕ2 (negative in this Embodiment). The tilt angle ϕ2 is obtained by arc tan (z2/y2)=arc tan {(z1+Δz1)/(y1+Δy1)}=arc tan {(Δz0+Δz1)/(y0+Δy0+Δy1)}.

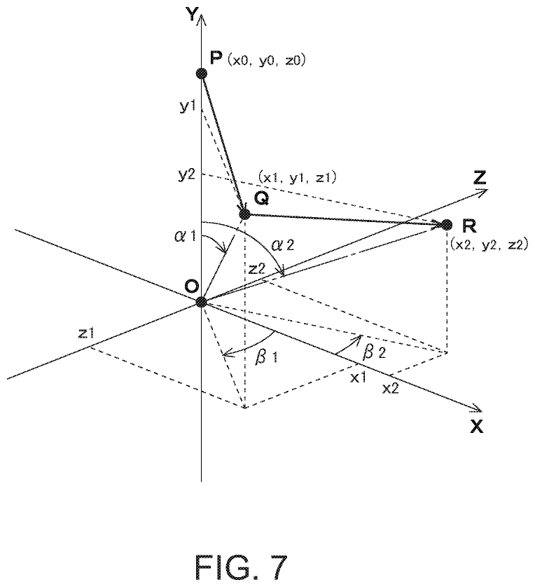

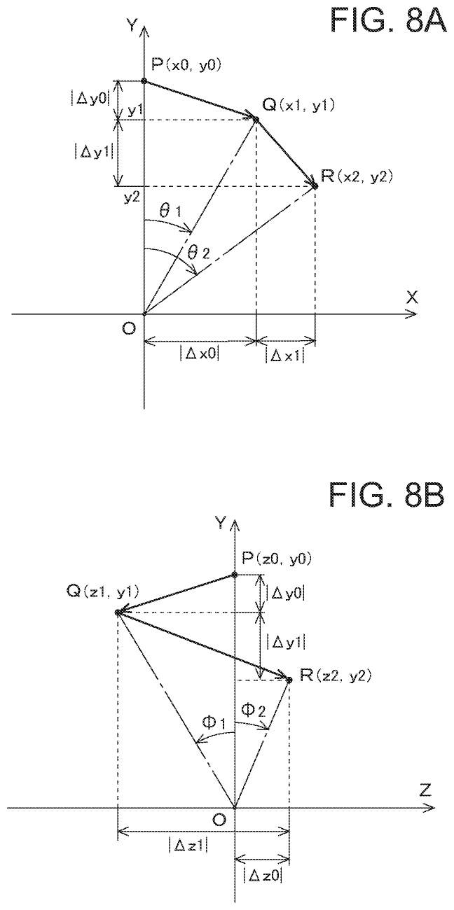

Further, in the case where movements of the head of the examinee from the point p to the point Q and point R include tilts to both of the back-and-forth direction (X-axis direction) and the right-and-left direction (Z-axis direction), the process in which the microcomputer 8 calculates a tilt angle in the point R will be described with reference to FIGS. 7, 8A and 8B. In FIG. 7, the tilt angle from the reference position P in the point R is calculated in three-dimensional space. In FIGS. 8A and 8B, as in FIGS. 5 and 6, by replacing the three-dimensional space with XY two-dimensional space and ZY two-dimensional space, the tilt angle from the reference position P in the point R is respectively calculated.

In FIG. 7, the point Q is a position of the head tilted from the reference position P in the back-and-forth and right-and-left directions, and the point R is a position of the head tilted from the point Q in the back-and-forth and right-and-left directions. The tilt angle from the Y axis in each point is a complementary angle of an elevation angle (positive in this Embodiment) in the Y-axis+direction from the XZ plane of each point, and it is assumed that the X-axis+direction from the Y axis is positive, and that the X-axis−direction is negative. In an azimuth angle of each point in the XZ plane, it is assumed that the Z-axis+direction (counterclockwise about the Y axis as the center) from the X axis is positive, and that the Z-axis−direction (clockwise about the Y axis as the center) is negative.

In FIG. 7, after tilting the head from the reference position P to the point Q in the X-axis+direction and Z-axis−direction, the head is tilted from the point Q to the point R in the X-axis+direction and Z-axis+direction. Herein, it is assumed that coordinates of the point Q are (x1, y1, z1), the tilt angle from the Y axis is α1, and that the azimuth angle from the X axis is β1. It is possible to obtain values of x1, y1 and z1, by respectively integrating accelerations in the X-axis, Y-axis and Z-axis directions acquired from the acceleration sensor 4 with respect to time, and thereby calculating moving distances Δx0, Δy0 and Δz0 from the reference position P to the point Q in the X-axis, Y-axis and Z-axis directions to add to the coordinates (x0, y0, z0)=(0, y0, 0) of the reference position P. Accordingly, the coordinates of the point Q are (Δx0, y0+Δy0, Δz0). In addition, in the moving distance of the head, it is assumed that the+direction in each of the X axis, Y axis and Z axis is the positive direction.

A length from the origin point O to the point Q is expressed by |OQ|=(x1²+y1²+z1²)^{1/2}={Δx0²+(y0+Δy0)²+Δz0²}^{1/2}=y0. Accordingly, the tilt angle α1 of the point Q is arc cos (y1/|OQ|)=arc cos (y1/y0)=arc cos (1+Δy0/y0). When the tilt angle α1 is a positive value, such an angle represents a tilt forward (X-axis+direction) from the reference position P. When the tilt angle α1 is a negative value, such an angle represents a tilt backward (X-axis−direction) from the reference position P. The azimuth angle β1 of the point Q is arc tan (z1/x1)=arc tan (Δz0/Δx0). When the azimuth angle β1 is a positive value, such an angle represents a tilt leftward (Z-axis+direction) from the reference position P in viewing the X-axis+direction as the front. When the azimuth angle β1 is a negative value, such an angle represents a tilt rightward (Z-axis−direction) from the reference position P in similarly viewing.

Next, when it is assumed that coordinates of the point R are (x2, y2, z2), it is possible to obtain values of x2, y2 and z2, by respectively integrating accelerations in the X-axis, Y-axis and Z-axis directions acquired from the acceleration sensor 4 with respect to time, and thereby calculating moving distances Δx1, Δy1 and Δz1 from the point Q to the point R in the X-axis, Y-axis and Z-axis directions to add to the coordinates (x1, y1, z1) of the point Q. In other words, it holds that x2=x1+Δx1=Δx0+Δx1, y2=y1+Δy1=y0+Δy0+Δy1, and that z2=z1+Δz1=Δz0+Δz1. Accordingly, the coordinates of the point R are (Δx0+Δx1, y0+Δy0+Δy1, Δz0+Δz1).

A length from the origin point O to the point R is expressed by |OR|=(x2²+y2²+z2²)^{1/2}={(Δx0+Δx1)²+(y0+Δy0+Δy1)²+(Δz0+Δz1)²}^{1/2}=y0. Accordingly, the tilt angle α2 of the point R is arc cos (y2/|OR|)=arc cos (y2/y0)=arc cos (y0+Δy0+Δy1)/y0. An azimuth angle β2 of the point R is arc tan (z2/x2)=arc tan {(Δx0+Δx1)/(Δz0+/Δz1)}. Similarly, the tilt angle α2 of a positive value represents a tilt forward (X-axis+direction) from the reference position P, and a negative value represents a tilt backward (X-axis−direction) from the reference position P. Further, the azimuth angle β2 of a positive value represents a tilt leftward (Z-axis+direction) from the reference position P in viewing the X-axis+direction as the front, and a negative value represents a tilt rightward (Z-axis−direction) from the reference position P in similarly viewing.

It is possible to obtain coordinates and tilt angle of each point in three-dimensional space of FIG. 7, by projecting into XY two-dimensional coordinates and ZY two-dimensional coordinates, and decomposing into the back-and-forth direction (X-axis direction) and the right-and-left direction (Z-axis direction). As in FIG. 5, FIG. 8A illustrates coordinates of each of points P, Q′ and R′ obtained by projecting each of points P, Q and R in the three-dimensional space into the XY two-dimensional coordinates. Herein, using the technique as described above in association with FIG. 6, coordinates of the point Q′ and point R′ are respectively calculated by integrating accelerations in the X axis and Y axis respectively obtained from the acceleration sensor 4 with respect to time.

In other words, when it is assumed that moving distances from the reference position P to the point Q′ in the X-axis and Y-axis directions are respectively Δx0 and Δy0, coordinates of the point Q′ are (x1, y1)=(Δx0, y0+Δy0). Similarly, when it is assumed that moving distances from the point Q′ to the point R′ in the X-axis and Y-axis directions are respectively Δx1 and Δy1, coordinates of the point R′ are expressed by (x2, y2)=(Δx0+Δx1, y1+Δy1)=(Δx0+Δx1, y0+Δy0+Δy1). Also herein, in the moving distance of the head, it is assumed that the+direction in each of the X axis and Y axis is the positive direction.

Accordingly, the tilt angle θ1 of the point Q′ is arc tan (x1/y1)=arc tan {Δx0/(y0+Δy0)}. The tilt angle θ2 of the point R′ is obtained by arc tan (x2/y2)=arc tan {(x1+Δx1)/(y1+Δy1)}=arc tan {(Δx0+Δx1)/(y0+Δy0+Δy1)}.

FIG. 8B illustrates coordinates of each of points P, Q″ and R″ obtained by projecting each of points P, Q and R in the three-dimensional space in FIG. 7 into the ZY two-dimensional coordinates. As described above in association with FIG. 6, coordinates of the point Q″ and point R″ are respectively calculated by integrating accelerations in the X axis and Y axis respectively obtained from the acceleration sensor 4 with respect to time. When it is assumed that moving distances from the reference position P to the point Q″ in the Z-axis and Y-axis directions are respectively Δz0 and Δy0, coordinates of the point Q″ are (z1, y1)=(Δz0, y0+Δy0). Similarly, when it is assumed that moving distances from the point Q″ to the point R″ in the Z-axis and Y-axis directions are respectively Δz1 and Δy1, coordinates of the point R″ are expressed by (z2, y2)=(Δz0+Δz1, y1+Δy1)=(Δz0+Δz1, y0+Δy0+Δy1). Also herein, in the moving distance of the head, it is assumed that the+direction in each of the Z axis and Y axis is the positive direction.

Accordingly, the tilt angle Φ2 of the point Q″ is arc tan (z1/y1)=arc tan {Δz0/(y0+Δy0)}. The tilt angle θ2 of the point R″ is obtained by arc tan (z2/y2)=arc tan {(z1+Δz1)/(y1+Δy1)}=arc tan {(Δz0+Δz1)/(y0+Δy0+Δy1)}.

In the above-mentioned Embodiment, as the method of converting coordinates of the point (the next point) subsequent to moving as a point that is directly moved from the reference position P when the measurement part (head) moves from some point to the next point in the three-dimensional space, in each coordinate system in FIGS. 5 to 8B, the method is described, assuming that the supporting point O to tilt the head is the origin point (0, 0, 0) of the coordinate axes, and that the reference position P of the measurement part is a point (X0, y0, z0)=(0, y0, 0) on the Y axis. In this case, in terms of calculation of the coordinates and tilt angle, it is desirable to beforehand determine the y0 value of the point P to a particular numeric value.

Generally, when the shoulder peak and the earhole are in the same vertical line viewed from the side, it is said that the head of a person is in a correctly upright posture, and tilts back and forth substantially with the shoulder peak as the center i.e. rotation supporting point. Accordingly, in this Embodiment, in the case where the acceleration sensor 4 is mounted in a head top position of an examinee, it is possible to determine a distance between the shoulder peak and the head top, by actually measuring the distance of the examinee in a state where the head is kept upright using a stadiometer and the like, or measuring from an image shot by a camera and the like. Further, for a body type of a person, it is also possible to estimate the distance between the shoulder peak and the head top of the examinee, by applying the height and size of the head of the examinee to previously stored data.

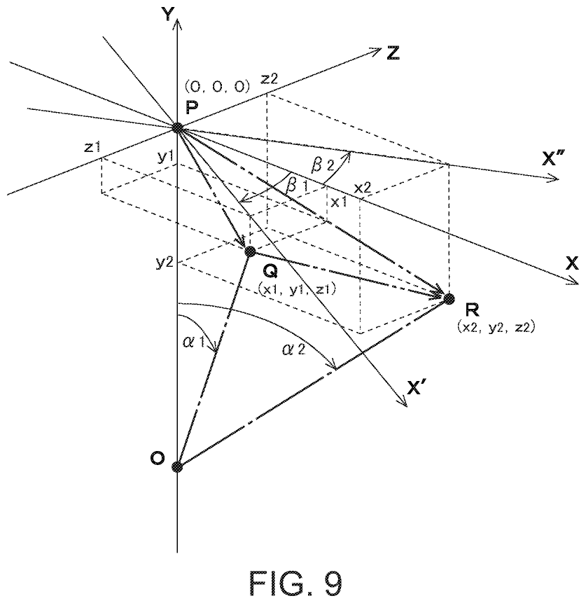

In another Embodiment, as shown in FIG. 9, by using an XYZ three-dimensional coordinate system with the reference position P of the measurement part as the origin point (0, 0, 0), it is possible to obtain coordinate positions and tilt angle of each point of the head moved from the reference position P from measured data of the acceleration sensor 4. In this case, there is no need for measuring the distance between the shoulder peak and the head top of the examinee, which is described above.

In FIG. 9, when it is assumed that coordinates of the point Q are (x1, y1, z1), values of x1, y1 and z1 are moving distances Δx0, Δy0 and Δz0 in the X-axis, y-axis and z-axis directions obtained by respectively integrating accelerations in the X-axis, Y-axis and Z-axis directions acquired from the acceleration sensor 4 in moving from the reference position P to the point Q with respect to time. Also in FIG. 9, in the moving distance of the head, the +direction of each of the X axis, Y axis and Z axis is assumed to be the positive direction.

Next, when it is assumed that coordinates of the point R are (x2, y2, z2), values of x2, y2 and z2 are obtained by adding moving distances Δx0, Δy0 and Δz0 in the X-axis, y-axis and z-axis directions, obtained by respectively integrating accelerations in the X-axis, Y-axis and Z-axis directions acquired from the acceleration sensor 4 in moving from the point Q to the point R with respect to time, to the coordinates (x1, y1, z1) of the point Q. In other words, it holds that x2=x1+Δx1=Δx0+Δx1, y2=y1+Δy1=Δy0+Δy1, and that z2=z1+Δz1=Δz0+Δz1.

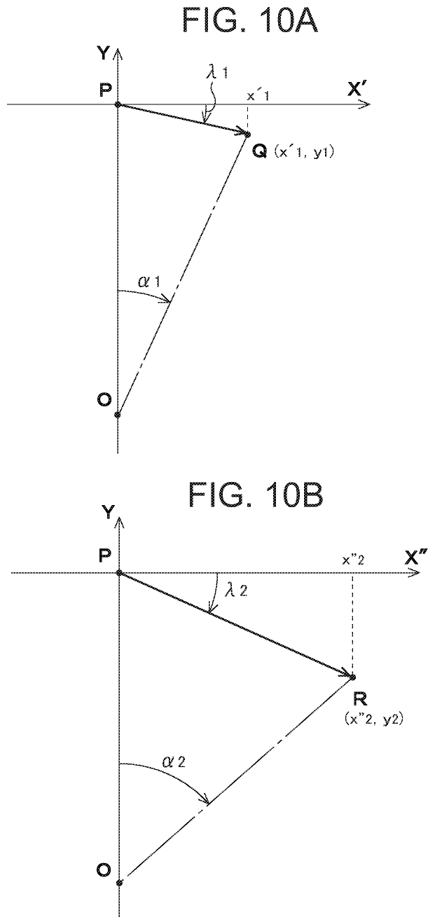

FIG. 10A is obtained by transferring the three-dimensional coordinates in FIG. 9 to two-dimensional coordinates of the plane including points 0, P and Q, and it is assumed that the straight line passing through the origin point P to be orthogonal to the Y axis is the X′ axis. In FIG. 10A, when it is assumed that coordinates of the point Q are (x′1, y1), it holds that x′1=(x1²+z1²)^{1/2}=(Δx0²+Δz0²)^{1/2}. The distance between the points P and Q is expressed by |PQ|=(x1²+y1²+z1²)^{1/2}=(Δx0²+Δy0²+Δz0²)^{1/2}.

When it is assumed that an elevation angle (angle from the X′ axis with the origin point P as the center) of the point Q is λ1, it holds that λ1=arc cos {(Δx0²+Δz0²)^{1/2}/(Δx0²+Δy0²+Δz0²)^{1/2}}. When it is assumed that the tilt angle of the point Q from the Y axis with the point O as the center is α1, the angle is expressed by α1=180° −2×(90°−λ1)=2λ1. Accordingly, the tilt angle α1 of the point Q is obtained by 2×arc cos {(Δx0²+Δz0²)^{1/2}/(Δx0²+Δy0²+Δz0²)^{1/2}}. Further, an azimuth angle β1 of the point Q in FIG. 9 is arc tan (z1/x1)=arc tan (Δz0/Δx0).

FIG. 10B is obtained by transferring the three-dimensional coordinates in FIG. 9 to two-dimensional coordinates of the plane including points O, P and R, and it is assumed that the straight line passing through the origin point P to be orthogonal to the Y axis is the X″ axis. In FIG. 10B, when it is assumed that coordinates of the point R are (x″2, y2), it holds that x″2=(x2²+z2²)^{1/2}={(Δx0+Δx1)²+(Δz0+Δz1)²}^{1/2}. The distance between the points P and R is expressed by |PR|=(x2²+y2²+z2²)^{1/2}={(Δx0+Δx1)²+(Δy0+Δy1)²+(Δz0+Δz1)²}^{1/2}.

When it is assumed that an elevation angle (angle from the X″ axis with the origin point P as the center) of the point R is λ2, it holds that λ2=arc cos [{(Δx0+Δx1)²+(Δz0+Δz1)²}^{1/2}/{(Δx0+Δx1)²+(Δy0+Δy1)²+(Δz0+Δz1)²}^{1/2}]. When it is assumed that the tilt angle of the point Q from the Y axis with the point O as the center is α1, the angle is expressed by α1=180° −2×(90°−λ1)=2λ1. Accordingly, the tilt angle α1 of the point Q is obtained by 2×arc cos [{(Δx0+Δx1)²+(Δz0+Δz1)²}^{1/2}/{(Δx0+Δx1)²+(Δy0+Δy1)²+(Δz0+Δz1)²}^{1/2}]. Further, an azimuth angle β2 of the point R in FIG. 9 is arc tan (z2/x2)=arc tan (Δz0+Δz1/Δx0+Δx1).

Thus, after setting the reference position P, the computation processing section 5 integrates the accelerations acquired from the acceleration sensor 4 every second with respect to time to calculate moving distances in the three-axis directions, and when a moving distance exists at least in one of the axes, based on the distance, calculates coordinates of a moved position. At this point, when there is no moving in any of the X axis, Y axis and Z axis, the computation processing section 5 continuously outputs coordinates that are detected last.

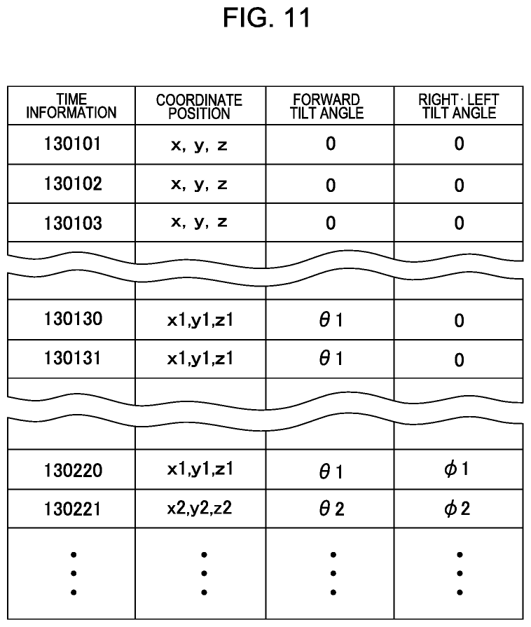

The microcomputer 8 associates the coordinates detected for each second and the tilt angle of the head forward calculated from the coordinates with the time information in the RTC 9 at this point, and stores the resultant in the memory 6. FIG. 11 conceptually illustrates a memory format of the memory 6, where coordinates of the head for each second, forward tilt angle, and tilt angle to the right or left are stored in time series throughout the time the examinee performs treatment.

Then, in the distortion detection system 1, when the USB port 10 is connected to the information processing apparatus 3 by USB, in response to instructions from the information processing apparatus 3, the computation processing section 5 reads the measured data and time information stored in the memory 6 to transmit.

The information processing apparatus 3 determines distortion of the posture of the examinee during treatment from the measured data read from the memory 6, and displays the result on a monitor screen using various graphs.

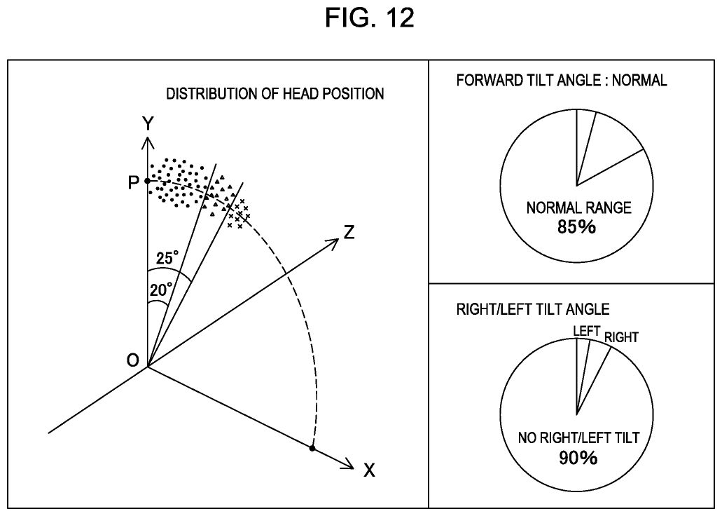

For example, as shown in FIG. 12, the apparatus 3 displays three-dimensional coordinate axes on the monitor screen, and plots each coordinate of the head throughout the treatment time to display. In this case, the apparatus determines coordinates falling within a proper range of 0 degree to 20 degrees as normal to display with green dots, determines coordinates in a range of 20 degrees or more and less than 25 degrees as caution needed to display with yellow “Δ” signs, and determines coordinates of 25 degrees or more as distortion to display with red “X” signs.

Then, the apparatus 3 displays a rate of coordinates belonging to each of the normal range, caution-needed range and distortion range in circle graph or bar graph, and according to the ratio, determines a distortion degree when the examinee tilts the head forward during the treatment. As the determination of the distortion degree, there is the case where the examinee brings the face near to an affected area and takes a posture of round shoulders to observe the affected area properly, and for example, when coordinates falling within the normal range are eight tenth or more, the normal posture is determined. Then, throughout the treatment time, the apparatus 3 displays a rate of time of taking the posture that the head is tilted to the left and right in the circle graph shown in the figure or bar graph.

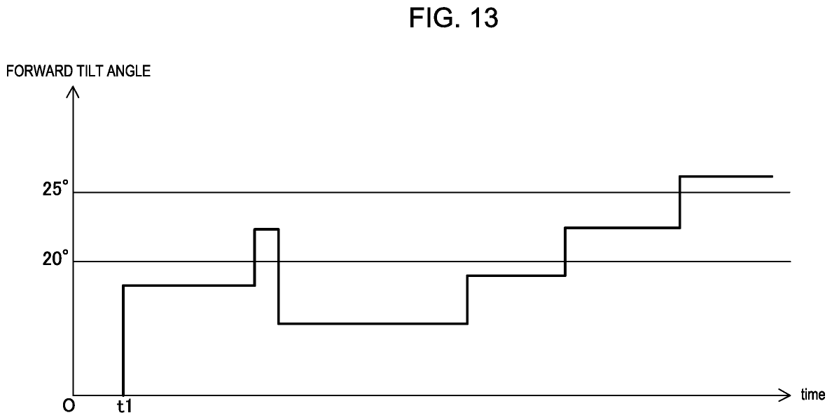

FIG. 13 illustrates an example for displaying changes in forward tilt angle of the neck in time series throughout treatment. In this case, a tl point in time is the time the examinee first tilts the neck forward after setting the reference position, and it is shown that the forward tilt angle is larger to distort the posture, as the time elapses.

In the above-mentioned Embodiment, the case is described where the acceleration sensor 4 of the distortion detection system 1 is disposed in the head top position of the examinee to use. In actual treatment work by a doctor, as shown in FIG. 14, it is considered that the acceleration sensor 4 is attached to the center of the bridge of the frame 16 of the binocular loupes 15. In this case, it is necessary to convert acceleration data acquired by the acceleration sensor 4 and/or calculation value of the moving distance to acceleration data and calculation value of the moving distance acquired in the head top position of the examinee. Since the relationship is certain between the bridge center position of the frame 16 of the binocular loupes 15 and the head top position of the examinee, such conversion is executable easily by a person skilled in the art, and specific calculation formulas are omitted.

In another Embodiment, the distortion detection system 1 is capable of being provided with a plurality of acceleration sensors 4. For example, in the binocular loupes 15 in FIG. 14, it is possible to arrange one of acceleration sensors 4a, 4b respectively on each of right and left temples 18. In this case, as described above, the posture of the head is in a state of being correctly upright when the shoulder peak and the earhole are in the same vertical line, and therefore, it is preferable that the sensor is provided in a position on the temple 18 extending upward perpendicularly directly from the earhole when the binocular loupes 15 are mounted.

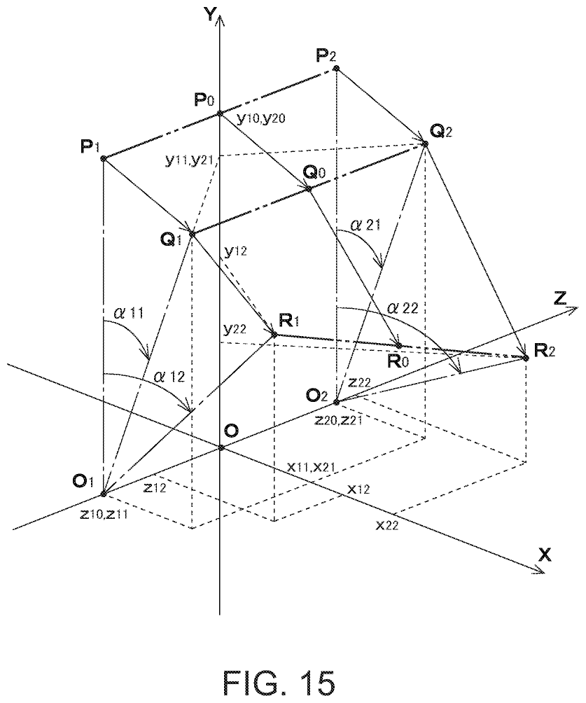

FIG. 15 illustrates coordinates in three-dimensional space of each measurement part moving in association with a movement of the head in the XYZ three-dimensional coordinate system, in the case where an examinee wears the binocular loupes 15 thus provided with two acceleration sensors 4a, 4b. In FIG. 15, points P1 and P2 are reference positions of measurement parts that correspond to the acceleration sensors 4a, 4b, respectively. Points Q1 and Q2 are positions of respective measurement parts moved from the reference positions P1 and P2, respectively, and points R1 and R2 are positions of respective measurement parts moved from the points P1 and P2, respectively.

Herein, it is assumed that coordinates of the points P1 and P2 are (x10, y10, z10) and (x20, y20, z20), coordinates of the points Q1 and Q2 are (x11, y11, z11) and (x21, y21, z21), and that coordinates of the points R1 and R2 are (x12, y12, z12) and (x22, y22, z22). It is the same as in each above-mentioned Embodiment that coordinates of the points Q1 and Q2 and points R1 and R2 are calculated by respectively integrating acceleration data acquired from the acceleration sensors 4a, 4b with respect to time, and adding the obtained moving distances to the coordinates of the points P1 and P2, and that tilt angles α11, α12, α21 and α22 of respective points are obtained from calculated coordinates of the points Q1 and Q2 and points R1 and R2, and therefore, descriptions thereof are omitted.

In FIG. 15, the distance between the points P1 and P2, the distance between the points Q1 and Q2, and the distance between the points R1 and R2 are always constant and the same. Accordingly, according to this Embodiment, it is possible to obtain the position relationship between the points Q1 and Q2 and the position relationship between the points R1 and R2 from the coordinates of each point and the tilt angle. As a result, with respect to the posture and movement of the head, as well as only the tilt in the back-and-forth direction and/or the right-and-left direction, it is possible to also grasp an extent of a twist (direction, level and the like thereof), and by adding the factors, it is possible to determine distortion of the body.

As mentioned above, described is the case of detecting distortion of the body by the tilt posture of the head during work, and it is also possible to detect another part of the body without being limited to the head. For example, in the posture in treatment by the doctor shown in FIG. 2, by winding a mount belt with the acceleration sensor 4 attached around the thigh part, upper arm or arm of the doctor, it is possible to detect coordinates of the part of the body with the acceleration sensor 4 mounted from the acceleration information.

Then, the information processing apparatus 3 is set to set a proper angle corresponding to the part of the body with the acceleration sensor 4 mounted. In other words, in the thigh part, the apparatus 3 sets angles in a range of 105 degrees to 125 degrees with respect to the center axis of the perpendicular body as normal. Accordingly, the information processing apparatus 3 displays the three-dimensional coordinate axes on the monitor screen, plots coordinates of the thigh part for each second throughout the treatment time to display, displays coordinates falling within the proper range of 105 degrees to 125 degrees with green dots, and displays coordinates falling outside the range with red “X” signs.

Further, the posture detection apparatus 2 may be provided with a biosensor for detecting the heart rate, respiration rate or temperature of the epidermis of the examinee, as well as the acceleration sensor 4. In this case, the posture detection apparatus 2 stores bio-information detected by the biosensor in the memory 6, as well as the time information of the RTC 9, and is thereby capable of making a determination by associating the bio-information read from the memory 6 with distortion.

### INDUSTRIAL APPLICABILITY

The present invention relates to the body distortion detection system for determining distortion of the body from daily action, and has industrial applicability.

1. A body distortion detection system including:
a posture detection apparatus mounted on the body of an examinee; and
a distortion determination apparatus,
wherein the posture detection apparatus is provided with a position detection sensor mounted on a part for detecting a movement of the body of the examinee to acquire position information of the part,
a computation processing section which calculates a moving distance of the part from the position information acquired periodically from the position detection sensor, and thereby obtains coordinates of the part, and
a memory that stores the coordinates obtained by the computation processing section point by point, and the distortion determination apparatus determines distortion of the body, based on a tilt angle of the part calculated from a series of the coordinates read from the memory.

2. The body distortion detection system according to claim 1, wherein the distortion determination apparatus reads the coordinates from the memory by USB connection with the posture detection apparatus.

3. The body distortion detection system according to claim 1, wherein the memory is a card type of flash memory capable of being removed from the posture detection apparatus to connect to the distortion determination apparatus.

4. The body distortion detection system according to claim 1, wherein the computation processing section and the memory are packaged with the position detection sensor to be mounted on the part.

5. The body distortion detection system according to claim 1, wherein the position detection sensor is mounted on the head of the examinee.

6. The body distortion detection system according to claim 1, wherein the position detection sensor is mounted on binocular loupes worn by the examinee.

7. The body distortion detection system according to claim 1, wherein the position detection sensor is an acceleration sensor or an angular velocity sensor.

8. The body distortion detection system according to claim 7, wherein the position detection sensor is comprised of a plurality of acceleration sensors or angular velocity sensors.

9. The body distortion detection system according to claim 1, wherein the posture detection apparatus is provided with a bio-sensor for detecting at least one of a heart rate, a respiration rate and a temperature of an epidermis of the examinee, and acquires bit-information based on detection output from the biosensor.
